(19) United States
(12) Patent Application Publication
(10) Pub. No.: US 2023/0166099 A1
(43) Pub. Date: Jun. 1, 2023

(54) Femoral venous and femoral arterial cannulas and a method for mitigating the risk of limb ischemia

(71) Applicant: TOTAL FLOW MEDICAL LTD, Vancouver (CA)

(72) Inventors: Luke Harris, Vancouver (CA); Andy Curtis, Vancouver (CA)

(73) Assignee: TOTAL FLOW MEDICAL LIMITED, Vancouver (CA)

(21) Appl. No.: 17/436,574

(22) PCT Filed: Feb. 13, 2020
(86) PCT No.: PCT/CA2020/050191

§ 371 Date: Sep. 3, 2021

(30) Foreign Application Priority Data
Mar. 5, 2019 (CA) 3035749

(51) Int. Cl. A61M 60/34; A61M 60/867; A61M 60/295; A61M 60/135; A61M 60/843; A61M 60/857

(52) U.S. Cl. CPC A61M 60/34; A61M 60/135; A61M 60/295; A61M 60/843; A61M 60/857; A61M 60/867; A61M 2025/0031

(57) ABSTRACT

A method and devices for mitigating the risk of limb ischemia includes inserting a femoral arterial cannula into a femoral artery of a limb, the femoral arterial cannula having a low-profile balloon to seal the femoral artery. Once in situ in the femoral artery, the femoral arterial cannula balloon is inflated to seal said femoral artery to deliver blood flow in a retrograde direction to said limb while simultaneously delivering systemic arterial blood flow. A femoral venous cannula is also inserted into a femoral vein of said limb, the femoral venous cannula having a broad-profile balloon to seal the femoral vein. Once in situ in said femoral vein, the femoral venous cannula balloon is inflated to seal the femoral vein to drain venous blood flow from the limb while simultaneously draining systemic venous blood flow.

### COPYRIGHT NOTICE

This patent document contains material which is subject to copyright protection. The copyright owner has no objection to the facsimile reproduction by anyone of this patent document as it appears in the Patent and Trademark Office patent file or records, but otherwise reserves all copyright rights whatsoever.

### FIELD OF THE INVENTION

The invention relates to the field of cannulas and more specifically to femoral and venous arterial cannulas and a method for mitigating the risk of limb ischemia. The invention provides femoral arterial and venous cannulas for cardiopulmonary support. Both cannulas utilize a balloon to isolate femoral artery and vein circulations from their respective systemic arterial and venous flow; subsequently these cannulas provide for dedicated femoral artery perfusion along with dedicated femoral vein drainage. The cannulas avoid the need to insert a distal limb perfusion cannula to facilitate arterial limb perfusion and therefore mitigate the complications associated therewith.

### BACKGROUND OF THE INVENTION

The use of extracorporeal membrane oxygenation (ECMO) in both acute and chronic based cardiac and respiratory failure is a world wide established and proven life saving measure. As reported by the Extracorporeal Life Support Organization, both the number of registered ECMO centers as well as the number of ECMO runs has dramatically increased since 1990. Consequently, the expected frequency of complications associated with such treatment has concomitantly and predictably increased. (www.ELSO.org)

ECMO is often fraught with a range of complications ranging in severity from bleeding to sepsis to death (Juo, et al. 2017). A major contributor to ECMO related morbidity and mortality is the development of the ischemic limb. The presence of a femoral arterial and venous cannula to achieve adequate cardiac output can significantly impair blood flow and venous drainage from the leg. This presents numerous challenges to the clinician. If not identified early, the consequences of limb ischemia are numerous, often requiring surgical intervention and challenges with blood pressure and hemodynamic patient management.

To facilitate access to the vascular system for various types of cardiopulmonary support it is necessary to gain access to both the arterial and venous system of a patient. It is a common procedure to position cannula in arteries and veins. These cannulas are designed to drain from and deliver blood to the circulation. There are alternative techniques beyond the use of a single arterial cannula, e.g. arterial graft anastomosed in a sideway manner to the femoral artery, dual arterial cannula, however the clinical status of the patient will often preclude the additional time required to facilitate such practice. In addition to this each and every additional intervention is associated with additional morbidities. There are on occasions clinical reasons to use more than one venous cannula however this is a practice more commonly associated with specific open-heart surgery procedures.

Positioned between these cannulas are various devices that facilitate and control blood flow (a mechanical pump) as well as gas and temperature exchange (mechanical oxygenator with an incorporated heat exchanger). There may or may not be various types of reservoirs and filters required, this is dependent upon the type of support necessary for the patient’s clinical status. These devices are connected to form a cardiopulmonary circuit with various lengths of tubing to join these multiple components.

Depending upon the reason for initiating cardiopulmonary support it is often necessary to introduce these cannulas into the femoral artery and femoral vein. This can be to expedite the institution of the necessary cardiopulmonary support (ECMO - Extracorporeal Membrane Oxygenation) or may be for an elective procedure due to the nature of the cardiac surgery that is to be performed, i.e. re-do OHS (open heart surgery), MICS (minimally invasive cardiac surgery), porcelain aorta, etc.

For multiple and complex clinical reasons the drainage from and delivery of blood to the ipsilateral limb into which these cannulas have been placed can be seen to be clinically inferior to the patient’s needs, i.e. inadequate arterial flow into the limb along with inadequate venous drainage from the limb.

The cause of these events is often mechanical in nature; the presence of the cannula within the lumen of the blood vessel can obliterate the blood pathway thus inhibiting the ability to either deliver blood downstream (retrograde) from the point of cannula insertion in the artery or to drain blood from the vein distal to the point of cannula entry.

Emergent cannulation of these vessels can often be traumatic resulting in damage to the vessel integrity along with vascular spasm. Limited evidence exists to suggest Body type and BMI are recognized as precipitators of limb mal-perfusion. The body of evidence strongly implies, however, that the ability to predict those susceptible to limb ischemia is highly ambiguous. This evidence highlights the need to institute both arterial and venous cannula designs shown below in all patients requiring ECMO or CPB. The use of dual arterial cannula (distal perfusion cannula) is complex and requires additional interventions; this is often precluded by the compromised nature of the patient’s cardiac status.

The incidence of limb ischemia is a common morbidity in the presence of femoral cannulation and is multi-factorial in nature. It is recognized that the diminished blood flow to the limb is significant in nature but is not the sole factor in precipitating limb ischemia. This feature when combined with reduced or negligible venous drainage from the limb renders it highly susceptible to the consequence of mal-perfusion.

Compromised venous drainage of the limb results in limb engorgement with an overall increase in vascular pressure. This increase subsequently impacts upon the ability to deliver arterial flow while also inhibiting lymphatic drainage.

The consequence of limb ischemia is significant. Venous thrombosis is a common morbidity, while tissue necrosis would be the ultimate sequelae. Further interventions are routinely required in the presence of limb ischemia such as embolectomies, fasciotomies and amputations.

Accordingly, a need exists for a novel cannulation design and a method of using the same to ensure adequate arterial flow and venous drainage is maintained to the limbs during peripherally cannulated ECMO, open heart surgery (OHS) and minimally invasive heart surgery (MICS). Other objects of the invention will be apparent from the description that follows.

### SUMMARY OF THE INVENTION

According to the present invention there is provided a femoral arterial cannula. The femoral arterial cannula may include an elongated hollow cylindrical cannula body having distal and proximal open ends. A low-profile balloon may be situated on the body adjacent the distal end and an inflation device may be connected to the balloon for inflating the balloon. Additionally, four blood flow exit ports may be situated in the cannula body between the balloon and the distal end with two of the distal exit ports in-line with one-another along the cannula body and two other distal exit ports being 180° circumferentially offset from the first two distal exit ports. Two blood flow exit ports may be situated in the cannula body between the balloon and the proximal end with the proximal exit ports being 180° circumferentially offset and longitudinally offset from one-another.

The cannula body may include a taper from the distal to the proximal end. The cannula body may also include a distal single radio-opaque marker and a proximal double radio-opaque marker.

The inflation device may include a small-bore tubing connected to the balloon at a first end, a balloon cuff connected to a second end of said small bore tubing, a two-way valve connected to the balloon cuff, and a female luer port connected to the two-way valve.

In accordance with another embodiment of invention there is provided femoral venous cannula. The femoral venous cannula may include an elongated hollow cylindrical cannula body of varying lengths having distal and proximal open ends. A broad-profile balloon may be situated on the body adjacent the proximal end and an inflation device may be connected to the balloon for inflating said balloon. Additionally, two blood flow entry ports may be situated in the cannula body between the balloon and the distal end with the distal entry ports being 180° circumferentially offset and longitudinally offset from one-another along the cannula body. Two blood flow entry ports may be situated in the cannula body between the balloon and the proximal end with the proximal entry ports being 180° circumferentially offset and longitudinally offset from one-another and 90° offset from the distal entry ports.

The cannula body may include a taper from said distal to said proximal end. The cannula body may also include a distal single radio-opaque marker and a proximal double radio-opaque marker.

The inflation device may include a small-bore tubing connected to said balloon at a first end, a balloon cuff connected to a second end of the small bore tubing, a two-way valve connected to the balloon cuff, and a female luer port connected to the two-way valve.

In accordance with yet another embodiment of invention there is provided a method for mitigating the risk of limb ischemia. The method may include inserting a femoral arterial cannula into a femoral artery of a limb with the femoral arterial cannula having a balloon to seal the femoral artery. Once in situ in the femoral artery, the method may include inflating the femoral arterial cannula balloon to seal the femoral artery to deliver blood flow in a retrograde direction to the limb while simultaneously delivering systemic arterial blood flow. Additionally, the method may include inserting a femoral venous cannula into a femoral vein of the limb with the femoral venous cannula having a balloon to seal the femoral vein. Once in situ in the femoral vein, the method may include inflating the femoral venous cannula balloon to seal the femoral vein to drain venous blood flow from the limb while simultaneously draining systemic venous blood flow.

Other aspects of the invention will be appreciated by reference to the detailed description of the preferred embodiment and to the claims that follow.

### BRIEF DESCRIPTION OF THE DRAWINGS

The preferred embodiment of the invention will be described by reference to the drawings thereof in which:

FIG. 1 is a femoral arterial cannula of the present invention;

FIG. 2 is a cross-sectional view along line 2-2 of FIG. 1;

FIG. 3 is a perspective view of the femoral arterial cannula of FIG. 1 with an inflated low-profile balloon;

FIG. 4 is the femoral arterial cannula of FIG. 1 inserted and in situ in a femoral artery;

FIG. 5 is an isolation of the tip of the femoral arterial cannula of FIG. 1 rotated 90 degrees;

FIG. 6 is a femoral venous cannula of the present invention;

FIG. 7 is a cross-sectional view along line 7-7 of FIG. 6;

FIG. 8 is a perspective view of the femoral venous cannula of FIG. 6 with an inflated broad-profile balloon;

FIG. 9 is the femoral venous cannula of FIG. 6 inserted and in situ in a femoral vein;

FIG. 10 is another embodiment the femoral venous cannula of the present invention; and

FIG. 11 is a cross-sectional view along line 10-10 of FIG. 10.

FIG. 12 is a schematic showing that institution of a distal limb perfusion cannula at the time of ECMO initiation is a superior solution to preventing limb ischemia versus as a rescue strategy (Lamb et al.)

FIG. 13 is an Illustration of a distal limb perfusion technique. Flow is provided to the ipsilateral limb via a branch off the ECMO circuit, which provides systemic flow. (From Lamb et al 2017)

### DESCRIPTION OF THE INVENTION

Ischemia is defined as, “an inadequate blood supply to an organ or part of the body”. Tissue injury and/or death occur as a result of the initial ischemic insult. The extent of injury is determined primarily by the magnitude and duration of the interruption in the blood supply. If unaddressed, cell death by necrotic, necroptotic, apoptotic, and autophagic mechanisms occurs (Kalogeris, et al. 2017).

Identification of limb ischemia caused by the presence of a femoral arterial cannula is of upmost importance with a universal recognition of the severity of consequences if not addressed early on. Given that this demographic of patients are most likely under general anesthesia the early signs of ischemia such as pain and paresthesia are often not possible to discern. This therefore relies on due diligence and frequent inspection of the patient’s limbs. Regular limb inspection is essential, assessing for pallor, reduced temperature, slow capillary refill and assessment of pulses via palpation or doppler.

More advanced stages of ischemia include mottled skin, increased limb circumference and compartment pressures above 30 mmHg. Accumulation of inflammatory mediators in a hypoxic environment in conjunction with the inability to wash out these mediators via the congested lymphatic system results in both apoptotic and necrotic cell death. This can cause an increase in systemic potassium. Both inflammatory mediators and high potassium can result in significant vasoplegia similar to that of sepsis. This often requires high dose vasopressors to maintain adequate systemic perfusion pressures. If distal limb flow is re-established late on in the ischemic insult the subsequent wash out of inflammatory mediators and potassium accumulation can further potentiate the vasoplegia (Kalogeris, et al. 2017).

Dependent on the extent at which the femoral artery and veins are occluded will determine the rate at which clinical signs for ischemia present themselves (Haley, et al.). This may be why ability to identify limb ischemia early on is fraught with difficulties. For example, a limb may present with reduced temperature and slow capillary refill, but integrity of pulses/flow are present via doppler and compartment pressures may be low. This may prompt a clinician to delay intervention given the potential for complications associated with the required intervention. The point at which intervention occurs therefore can vary significantly. Given the difficulty to reliably assess limb ischemia and varying degrees at which identifiable signs present themselves explains in part why some institutions consistently place a distal limb catheter as a prophylactic measure to ensure adequate limb flow is maintained.

### (ECMO) and Limb Ischemia Reporting and Incidence

The incidence of reported limb ischemia in ECMO patients seems to vary significantly across ECMO centers.

The North American 2017 ELSO report on Adult V-A ECMO complications shows a 5.5% incidence of limb ischemia with a 21% survival rate.

This incidence seems low compared to publications on limb ischemia. A review of reported limb ischemia incidence by Lamb et al. 2016 et al. reported a range of limb ischemia events from 10-70% of patients that receive peripherally cannulated ECMO. In a meta analysis of limb ischemia incidence (Juo et al.2017) reported limb ischemia in 17% of patients out of a total 1886 patients. Possible reasons for discrepancies in reporting maybe due to compounding complications and primary causes of mortality rates in ECMO patients. The potential for limb ischemia being unidentified or shadowed by other causes of mortality may result in under reporting. Furthermore, there may also be significant differences in cannulation technique, size of cannula, point at which the cannula is inserted, native anatomy nuances as well as the timing with which distal limb catheters are inserted.

It is however noted that the presence of limb ischemia in the absence of other ECMO related complications is devastating and considered an independent risk factor for mortality (Juo et al.)

TABLE 1

Patient demographics taken from Yuo et al.2016. This table demonstrates that expected co-morbidities that are expected to increase likelihood of limb ischemia are non-correlating.
Patient Demographics

| Variable | Total (N=151) | Rescue strategy (N = 107) | Preemptive strategy (N =44) | P |
|---|---|---|---|---|
| Age (years) | 54.6 ± 16.3 | 57.2 ± 15.1 | 48.1 ± 17.4 | 0.003 |
| Male | 90 (59.6) | 64 (59.8) | 26 (59.1) | 0.935 |
| Height (m) | 1.6 ± 0.1 | 1.6 ±0.1 | 1.6 ± 0.1 | 0.985 |
| BSA | 1.7±0.2 | 1.7 ± 0.2 | 1.6 ±0.1 | 0.291 |
| BMI | 23.2 ±4.0 | 23.6 ±4.1 | 22.4 ± 3.6 | 0.102 |
| APACHE II | 13.0 ± 6.3 | 12.2 ±6.0 | 14.8 ±6.9 | 0.026 |
| SOFA | 11.6 ±3.5 | 11.7 ±3.5 | 11.3 ±3.3 | 0.585 |

| | | | | |
|---|---|---|---|---|
| Ischemia risk factor | | | | |
| Diabetes | 25 (16.4) | 21 (19.6) | 4 (9.1) | 0.113 |
| Hypertension | 39 (25.7) | 32 (29.9) | 7 (15.9) | 0.074 |
| Chronic renal insufficiency | 6 (3.9) | 5 (4.7) | 1 (2.3) | 0.493 |
| Smoker | 27 (17.8) | 20 (18.7) | 7 (15.9) | 0.685 |
| PVD | 11 (7.2) | 9 (8.4) | 2 (4.5) | 0.406 |
| Cerebral infarction | 5 (3.4) | 5 (4.8) | 0 | 0.139 |
| Catheter size (Fr.) | 17.4 ±2.0 | 17.2 ±2.1 | 17.9 ±1.8 | 0.059 |
| Diameter of catheter (mm) | 5.8 ±0.7 | 5.7 ±0.7 | 6.0 ± 0.6 | 0.052 |
| Cannula size (Fr.)-to-BSA ratio | 10.6 ±1.5 | 10.4 ±1.6 | 11.0 ± 1.2 | 0.056 |
| Diameter of cannula (mm)-to-BSA ratio | 3.5 ±0.5 | 3.5 ± 0.5 | 3.7 ± 0.4 | 0.050 |
| ECMO days | 5.2 ±5.0 | 4.9 ±4.9 | 6.0 ±5.4 | 0.212 |
| Vasopressor use | 44 (29.1) | 29 (27.1) | 15 (34.1) | 0.390 |
| Dialysis | 46 (30.3) | 33 (30.8) | 13 (29.5) | 0.875 |
| Aspirin use | 29 (19.1) | 19 (17.8) | 10 (22.7) | 0.48 1 |
| Dual anti-platelet drug use | 23 (15.1) | 17 (15.9) | 6 (1 3.6) | 0.726 |
| Warfarin use | 2 (1.3) | 1 (0.9) | 1 (2.3) | 0.513 |
| Successful weaning | 98 (64.9) | 61 (57.0) | 37 (84.1) | 0.002 |
| Overall mortality | 83 (55.0) | 66 (61.7) | 17 (38.6) | 0.010 |

BSA, body surface area; BMI, body mass index; PVD, peripheral vascular disease; APACHE 11, Acute Physiology and Chronic Health Evaluation II score; SOFA, Sequential Organ Failure Assessment score; ECMO, extracorporeal membrane oxygenation. Data are presented as numbers (with percentages) or as means ± standard deviation.

### Risk Factors for Limb Ischemia

The identification of risk factors for the generation of limb ischemia is highly ambiguous. Presumed risk factors such as presence of obesity, diabetes, peripheral vascular disease and age does not seem to correlate with a higher incidence of limb ischemia. This is noted by Gander et al (2010) who experienced 52% incidence of limb ischemia and noted that, “No variable was predictive of the development of significant limb ischemia”. It could be stipulated that the inherent risk could be solely due to the diameter of the femoral artery in relation to the size of the femoral arterial cannula as well as the point of insertion.

Conversely, the development of limb ischemia could be more complex than solely reduced arterial flow to the limb. This is noted by the prevalence of reported limb ischemia in Veno-Venous ECMO via ELSO.org. This suggests that the generation of limb ischemia could also be caused by venous and lymphatic congestion due to the presence of the femoral venous cannula.

Arguably it appears that there are a myriad of factors that result in limb ischemia which will likely explain why there are varying degrees of reported ischemic insults and degrees of success when surgical intervention and institution of a distal limb perfusion catheter is inserted.

This is supported by Yuo et al.2016 who examined the difference between ECMO patients that received immediate placement of a distal limb perfusion catheter and those who had one inserted as a rescue strategy at the first signs of limb ischemia.

The table 2 from Yuo et al.2016 demonstrates that a rescue strategy was not as successful as a pre-emptive strategy. The patients that received a distal limb catheter at the time of ECMO initiation had zero incidence of limb ischemia and therefore required no further surgical intervention such as fasciotomy or amputation. It is also notable that the difference in mortality was significantly

TABLE 2

The Cannula-Related Complications Developing During ECMO Support

| | Total (N=151) | Rescue strategy (N =107 | Preemptive Strategy (N =44) | P |
|---|---|---|---|---|
| Ischemia Surgical intervention Fasciotomy Amputation Cannulationsite bleeding | 10 (6.6) 3 (2.0) 2(1.3) 1(0.7) 14(9.3) | 10 (9.3) 3 (2.8) 2 (1.9) 1 (0.9) 9(8.4) | 0 0 0 0 5 (11.4) | 0.036 0.262 0.359 0.248 0.570 |

Data are presented as numbers (with percentages).

lower in the preemptive strategy. Table 2: Yuo et al.2016 demonstrating the difference between a rescue and preemptive strategy to address ischemic limb as a consequence to peripherally cannulated ECMO.

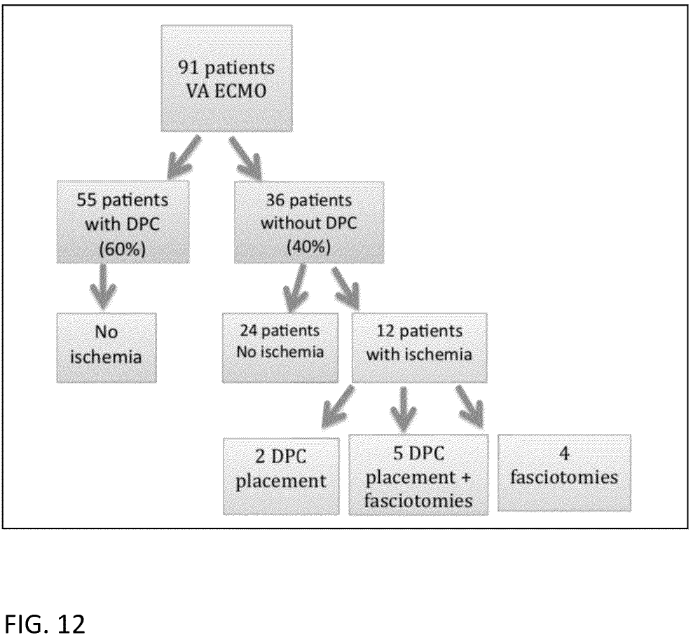

This finding was also found by Lamb et al. 2017 who through examination of their ECMO patients demonstrated that immediate institution of a distal limb cannula prevents the development of limb ischemia in 100% of their patient’s versus those who did not receive a distal limb cannula where 50% developed ischemic limb complications that required intervention including fasciotomies. (See FIG. 12)

### Distal Limb Insertion Techniques and Complications

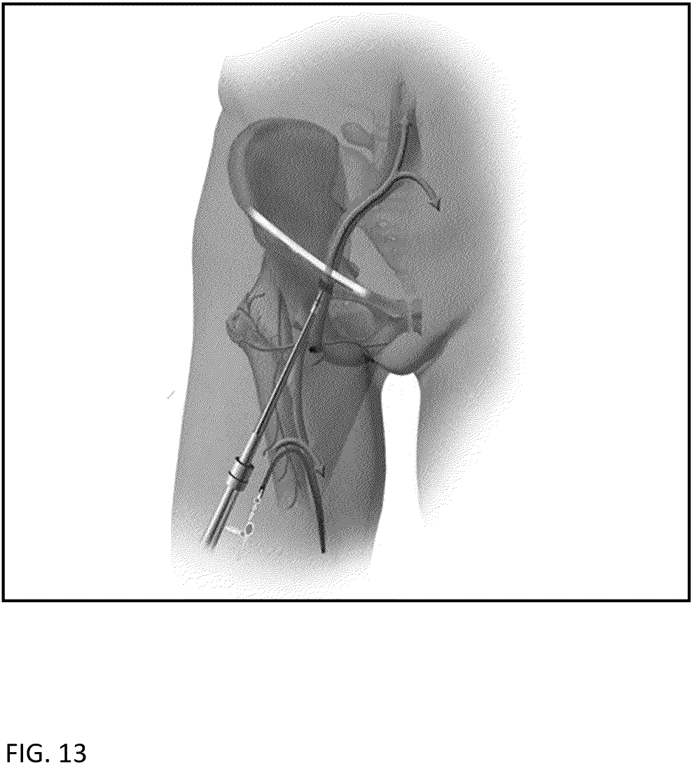

The insertion of a distal limb perfusion cannula as shown in FIG. 13 (which is an Illustration of a distal limb perfusion technique, wherein flow is provided to the ipsilateral limb via a branch off the ECMO circuit, which provides systemic flow. (From Lamb et al 2017)_is not without complication. Lamb et al. 2017 notes that femoral vessel complications associated with cannulation include arterial dissection, pseudo-aneurysms, thromboembolic complications and infectious arterial complications. It could be surmised that these complications are inclusive of placement of a distal limb cannula.

Furthermore the manner with which flow to the limb is incorporated into the ECMO circuit can expose the patient to potential hazards and requires due care and attention by the Perfusionist to ensure the integrity of the ECMO circuit is maintained.

Given that the limb perfusion cannula provides flow via extra ¼ inch tubing either via a Y connector off the main ⅜ inch tubing or an external port on the ECMO oxygenator, if the flow is not sufficient it can often result in clot formation and potentially embolic delivery to the limb. This can often compound limb ischemia and requires complex intervention by a vascular surgeon to perform embolectomies and or femoral bypass grafts. This can further expose the patient to excessive bleeding, potential for infection as well as increased blood transfusion rates. It is not uncommon for ECMO patients to receive multiple allogeneic blood transfusions. Given this patient demographic can be often worked up for heart transplant candidacy, increased blood transfusions can result in sensitizing the humoral immune system making it increasingly difficult to find a suitable, compatible donor. Therefore limiting allogeneic blood transfusions is essential.

### Venous and Lymphatic Congestion

The etiology of lymphatic congestion is a complex process, which is not fully understood.

The relationship between arterial, venous, interstitial and lymphatic blood flow is a dynamic complex balance which can be easily influenced by osmotic and oncotic changes as well as blood flow rates. It is important to note that lymph transport, not venous capillary reabsorption, is the main process responsible for interstitial fluid drainage (Mortimer and Levick 2004). Therefore all edema is due to an imbalance between capillary filtration and lymph drainage. It is theorized therefore that in the presence of a femoral venous cannula, reduction in venous drainage from the limb results in subsequent venous engorgement. This then results in increased capillary perfusion pressures with subsequent fluid movement from the venous compartment to the lymphatic vessels. It is thought that the ability of the lymphatic compartment to compensate for this increased interstitial blood flow is low and limb edema ensues. If this is paired with low oncotic pressures (fluid overload is common in patients requiring ECMO as well as Cardiopulmonary bypass for OHS/MICS) increased capillary filtration then overwhelms increased lymph drainage, which subsequently under-compensates.

Interstitial edema in this overwhelmed vascular compartment then increases the oxygen diffusion barrier resulting in cellular ischemia. The inflammatory process then further exacerbates the interstitial edema due to the development of “leaky” capillary beds.

It is believed that this is an under appreciated cause of limb ischemia in peripherally cannulated ECMO and CPB (OHS/MICS) patients and highlights the need to not only maintain arterial blood flow but also venous drainage and therefore prevent overwhelming the lymphatic compartments during the period of cardiopulmonary support. This may also explain why ischemic limb has been seen as a complication in Veno-venous ECMO patients where no arterial cannula is placed.

An object of the inventions is to mitigate the potential for limb ischemia occurring as a consequence to the presence of arterial and venous femoral cannulation. The cannulas are designed to require no additional components other than that of the arterial and venous cannula themselves. The cannulas are designed to be suitable for all femoral cannulation requirements, be it for ECMO, OHS, MICS or other cardiopulmonary systems.

The function of the femoral arterial cannula is to provide retrograde arterial limb flow in the presence of systemic arterial flow via a single cannula.

The function of the femoral venous cannula is to provide retrograde venous limb drainage in the presence of systemic venous flow via a single cannula.

### Femoral Arterial Cannula

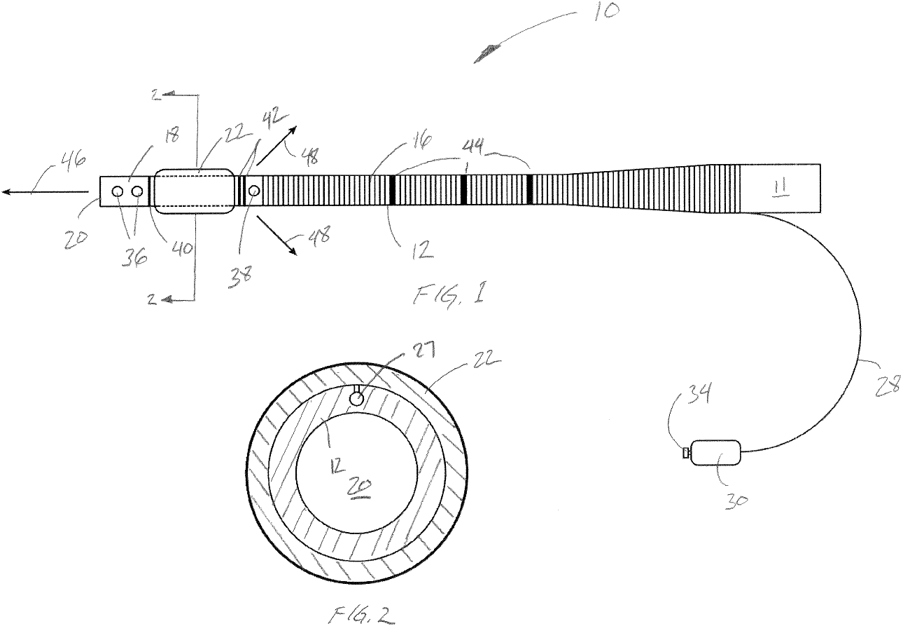

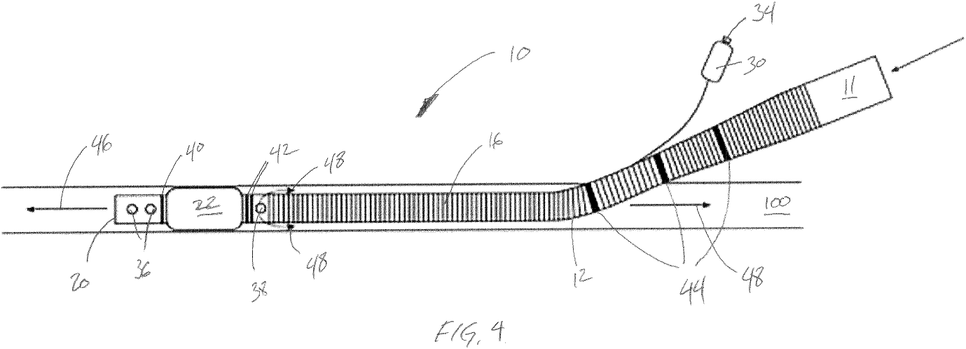

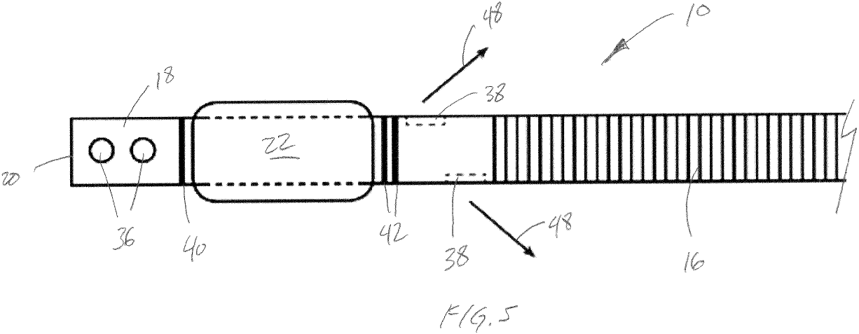

Referring to FIGS. 1 to 5 a femoral arterial cannula 10 features a cylindrical plastic body of tubing 12 which preferably has a biocompatible surface coating. The initial length 11 (⅜″ OD) remains clear and a ⅜x⅜″ luer lock connector is used to connect the initial length with a cardiopulmonary circuit; arterial blood flow enters the cannula 10 through this connector. This connector is a standard perfusion component and is not shown in the figures.

The length of body tubing 12 tapers to one of the varied sizes; e.g. a 15 Fr (5.0 mm) body of wire wound tubing 16 or a 17 Fr (5.6 mm) body of wire wound tubing or a 19 Fr (6.3 mm) body of wire wound tubing 16.

Distal to the length of wire wound tubing 16 is a contiguous length of clear tubing 18, the tip 20 of which is open ended and is the exit port for systemic arterial flow.

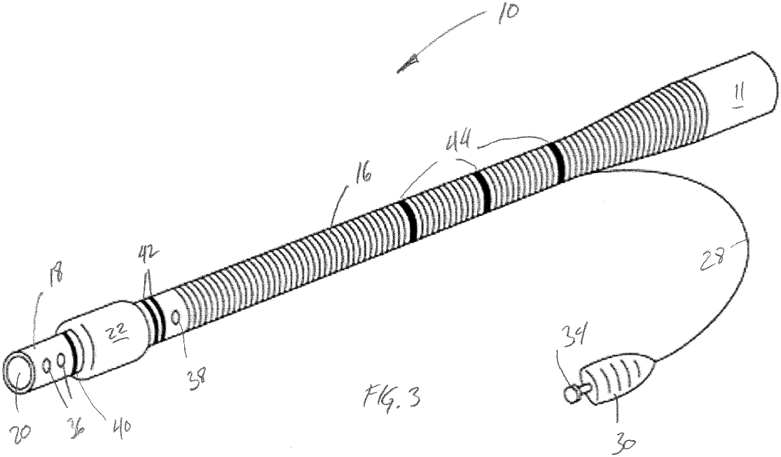

Circumferentially attached to body tubing 12 is a low-profile compliant balloon 22. Balloon 22 remains parallel to the length of clear tubing 12 when non-inflated. The balloon 22 length is approximately 20 mm. The external balloon 22 surface has a drug eluting coating. When inflated the balloon 22 remains cylindrical along its length and is parallel to the body 12 of the cannula 10. A channel 27, as best illustrated in FIG. 2, runs the length of body tubing 12 to accommodate and allow a small-bore length of tubing 28 to sit longitudinally flush along body tubing 12. Small-bore length tubing 28 is attached to balloon 22 at one end and at its other end, to a balloon cuff 30 that includes a two-way valve and female luer port 34.

Distal to the distal end of the balloon 22 are four circular blood pathway exit ports 36 within contiguous length of clear tubing 18; as illustrated, two exit ports longitudinally with identical blood flow exit ports at 180 degrees circumferentially. Exit ports 36 are contiguous with the blood pathway for systemic arterial flow.

Proximal to the proximal end of the balloon 22 are two exit ports 38 within the contiguous length of clear tubing 18; exit ports 38 are at 180 degrees circumferentially, and as best illustrated in FIG. 5, are offset longitudinally from each other by 5 mm (one exit port is 5 mm from the proximal balloon end while the second exit port is 10 mm from the proximal balloon end).

Distal to the distal end of the balloon 22 is a circular radio-opaque marker 40 within the body tubing 12. Proximal to the proximal end of the cannula 10 are two parallel circular radio-opaque markers 42 within the body tubing 12. Positional markers 44 are identified on the length of the wire wound tubing 16 body of the body tubing 12 to identify cannula positional depth.

The femoral arterial cannula 10 is designed to be inserted either by a surgical cut down or by a percutaneous Seldinger technique. The cannula 10 delivers systemic arterial blood flow 46 along the length of the cannula 10 body through to the cannula tip 20 and the additional blood pathway exit ports 38. The balloon 22 is designed to seal the femoral artery 100 once inflated. The balloon 22 has a drug eluting coating. The cannula 10 has a biocompatible surface coating. The balloon 22 is de-aired prior to insertion by means of injection and aspiration of saline/contrast solution. Once in situ and inflated the balloon 22 will effectively isolate the blood flow of the proximal exit ports 38 such that they are only available to deliver flow in a retrograde direction down 48 to the ipsilateral limb. With the balloon 22 inflated there is dedicated limb blood flow delivery 48.

### Femoral Venous Cannula

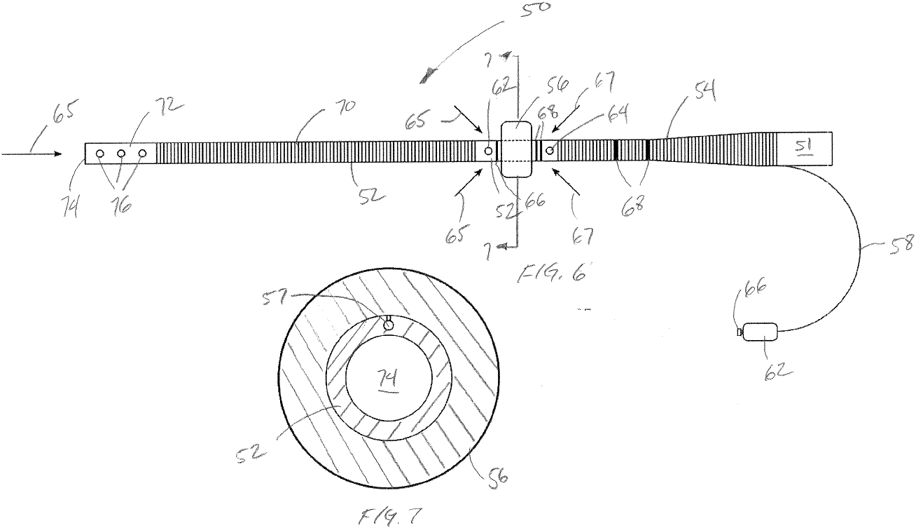

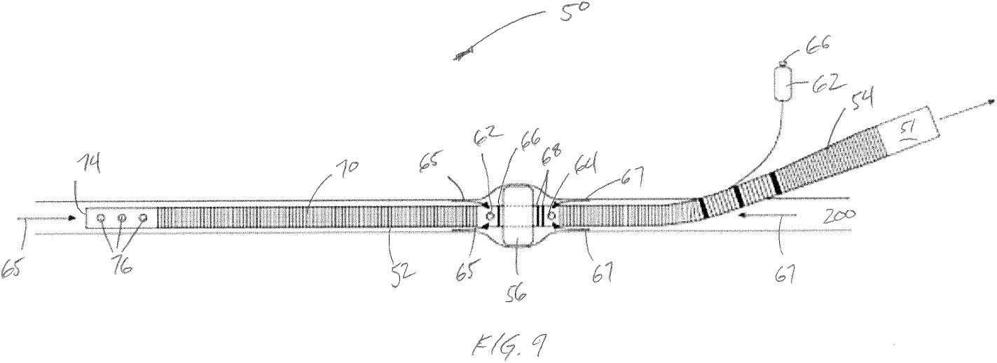

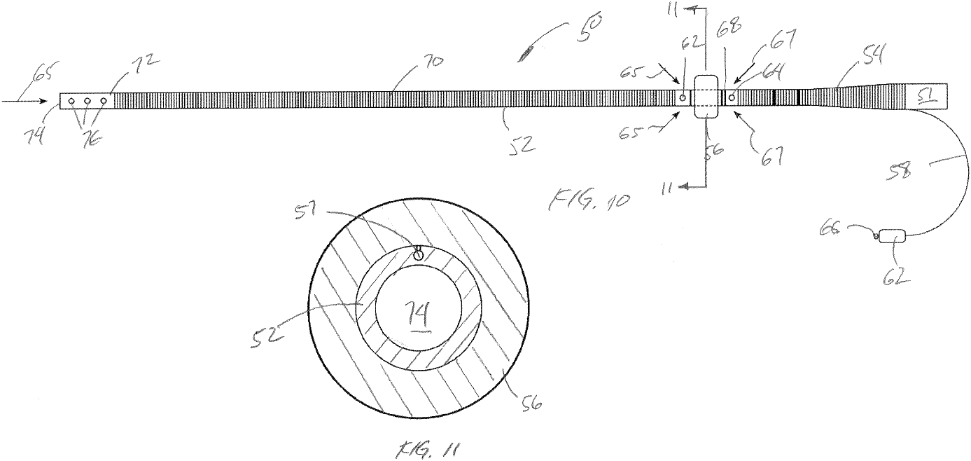

Referring to FIGS. 6 to 11, the femoral venous cannula 50 features a cylindrical plastic length of tubing 52 which preferably has a biocompatible surface coating. The initial length 51 (⅜″OD) remains clear with a ⅜x⅜ connector in situ (non luer lock); venous blood flow exits the cannula 50 through this connector which is a standard perfusion item and not shown in the figures. This length of tubing 52 tapers to one of varied sizes: a 23 Fr (7.6 mm) body of wire wound tubing 54 or a 25 Fr (8.3 mm) body of wire wound tubing or a 27 Fr (9.7 mm) body of wire wound tubing..

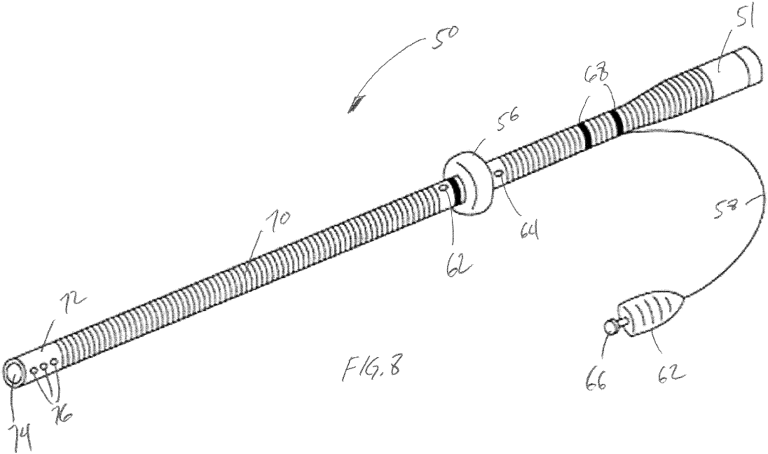

Distal to the length of wire wound tubing 54 is a contiguous length of clear tubing 52. Attached to clear length of tubing 52 is a broad-profile compliant balloon 56 which remains parallel to the length of clear tubing 52 when non-inflated. The external balloon 56 surface has a drug eluting coating. The balloon 56 length is approximately 12 mm. When inflated the balloon 56 remains cylindrical along its length and is parallel to the body tubing 52. A channel 57, as best illustrated in FIGS. 7 and 11, runs the length of body tubing 52 to accommodate and allow a small-bore length of tubing 58 to sit longitudinally flush along body tubing 52. Small-bore length tubing 58 is attached to balloon 56 at one end and at its other end, to a balloon cuff 62 that includes a two-way valve and female luer port 66.

Distal to the distal end of the balloon 56 are two circular blood pathway entry ports 62 within the clear tubing 52; the two blood entry ports 62 are at 180 degrees circumferentially. These blood entry ports 62 are offset from each other by 5 mm (one entry port is 5 mm distal from the balloon 56 while the second entry port is 10 mm from the balloon 56 end). These entry ports 62 are contiguous with the blood pathway 65 for systemic venous blood flow.

Proximal to the proximal end of the balloon 56 are two blood pathway entry ports 64 within the clear tubing 52, these ports are at 180 degrees circumferentially but are off-set from each other by 5 mm (one entry port is 5 mm proximal from the balloon 56 end while the second entry port is 10 mm from the balloon end). These ports 64 are circumferentially offset by 90 degrees from the two distal blood entry ports 62.

Distal to the distal end of the balloon 56 is a single circular radio-opaque marker 66 within the body of the cannula 50. Proximal to the proximal end of the cannula 10 are two parallel circular radio-opaque markers 68 within the body tubing 52.

Distal to the clear tubing 52 is a contiguous length of wire wound tubing 70. Distal to the length of wire wound tubing 70 is a contiguous length of clear tubing 72, the tip 74 of which is open ended and is the entry port for systemic venous flow 65. Within this length of clear tubing 72 are six circular blood pathway entry ports 76 within the clear tubing 72, three entry ports longitudinally with identical exit ports at 180 degrees circumferentially.

Positional markers 68 are identified on the length of the wire wound tubing body 54 of the body tubing 52 to identify cannula positional depth.

The femoral venous cannula 50 is designed to be in varying lengths depending upon its clinical application as well the physical size of the patient. The femoral venous cannula 50 that has application for V-A ECMO, OHS and MICS procedures has three lengths while the model for V-V ECMO has three lengths.

The femoral venous model designed for V-V ECMO is a shorter length than that of the femoral venous cannula designed for V-A ECMO, OHS and MICS. The included schematic has individual design components identical to those of the longer cannula.

The femoral venous cannula 50 is designed to be inserted either by a surgical cut down or by a percutaneous Seldinger technique. The cannula 50 drains systemic venous blood flow 65 along the length of the body tubing 52 from the cannula tip 74 and the additional blood pathway entry ports 62. The balloon 56 is designed to seal the femoral vein 200 once inflated. The balloon 56 has a drug eluting coating. The cannula 50 has a biocompatible surface coating. The balloon 56 is de-aired prior to insertion by means of injection and aspiration of saline/contrast solution. Once in situ and inflated the balloon 56 will effectively isolate the blood flow of the proximal entry ports 64 such that they are only available to drain venous blood flow 67 from the ipsilateral limb. With the balloon 56 inflated there is dedicated limb flow drainage while the distal entry ports 62 directly above the balloon will drain venous blood from the lower length of the inferior vena cava.

While it is recognized that either cannula can be used individually, the central concept to completely address and prevent limb ischemia is to use both arterial 10 and venous 50 cannula to work synergistically.

### Method of Mitigating Risk of Limb Ischemia

The femoral arterial 10 and venous 50 cannulas have been designed to facilitate the delivery of dedicated arterial limb flow in the presence of dedicated venous limb drainage. A non-pferfused limb will suffer the inevitable clinical consequences of ischemia, however a limb that is adequately perfused but is insulted by having inadequate or absent venous drainage will similarly be subjected to a physiological insult that may ultimately be irrecoverable. By facilitating both arterial perfusion along with venous drainage will protect the limb from the adverse consequences of limb ischemia. This identifies and recognizes the multifactorial nature of ischemic limb associated with peripheral cannulation and therefore offers a clinically superior solution opposed to using one cannula in isolation.

While embodiments of the invention have been described and illustrated, such embodiments should be considered illustrative of the invention only. The invention may include variants not described or illustrated herein in detail. Thus, the embodiments described and illustrated herein should not be considered to limit the invention as construed in accordance with the accompanying claims.

### References

Theodosios Bisdas, Gernot Beutel, Gregor Warnecke, Marius M. Hoeper, Christian Kuehn, Axel Haverich, Omke E. Teebken. 2011.Vascular Complications in Patients Undergoing Femoral Cannulation for Extracorporeal Membrane Oxygenation Support. 92:626-31. Ann Thorac Surg.

Jeffrey W. Gander* , Jason C. Fisher, Ari R. Reichstein, Erica R. Gross, Gudrun Aspelund, William Middlesworth, Charles J. Stolar. 2010. Limb ischemia after common femoral artery cannulation for venoarterial extracorporeal membrane oxygenation: an unresolved problem. 45, 2136-2140. Journal of Pediatric Surgery.

Yi Juo , Matthew Skancke, Yas Sanaiha, Aditya Mantha, Juan C. Jimenez, Peyman Benharash. 2017. Efficacy of Distal Perfusion Cannulae in Preventing Limb Ischemia During Extracorporeal Membrane Oxygenation: A Systematic Review and Meta-Analysis. 41(11):E263-E273 Artificial Organs.

Mary Jo Haley, Jason C. Fisher, Alejandro R. Ruiz-Elizalde, Charles J.H. Stolar, Nicholas J. Morrissey, William Middlesworth. 2008. Percutaneous distal perfusion of the lower extremity after femoral cannulation for venoarterial extracorporeal membrane oxygenation in a small child.44, 437-440. Journal of Pediatric Surgery

Theodore Kalogeris, Christopher P. Baines, Maike Krenz, and Ronald J. Korthuis. 2012. Cell Biology of Ischemia/Reperfusion Injury. 298: 229-317. Int Rev Cell Mol Biol.

Kathleen M. Lamb, Hitoshi Hirose. 2017. Vascular Complications in Extracoporeal Membrane Oxygenation. 33, 813-824. Crit Care Clin.

Kathleen M. Lamb, Paul J. DiMuzio, Adam Johnson, Philip Batista, Neil Moudgill, Megan McCullough, Joshua A. Eisenberg, Hitoshi Hirose, MD, Nicholas C. Cavarocchi. 2016. Arterial protocol including prophylactic distal perfusion catheter decreases limb ischemia complications in patients undergoing extracorporeal membrane oxygenation.65:1074-9. Society for Vascular Surgery.

Lukasz A. Adamczyk’ Kristiana Gordon’ Ivana Kholová’ Lorine B. Meijer-Jorna′

Niklas Telinius’ Patrick J. Gallagher’ Allard C. van der Wal′ Ulrik Baandrup. 2016. Lymph vessels: the forgotten second circulation in health and disease. 469:3-17. Virchows Arch.

Hye Ju Yeo, Seong Hoon Yoon, Doosoo Jeon, Yun Seong Kim, Woo Hyun Cho, Dohyung Kim, Seung Eun Lee. 2016. The Utility of Preemptive Distal Perfusion Cannulation During Peripheral Venoarterial Extracorporeal Membrane Oxygenation Support. Vol. 29, No. 4. Journal of interventional cardiology.

What is claimed is:

1. A method for mitigating the risk of limb ischemia comprising:
inserting a femoral arterial cannula into a femoral artery of a limb, said femoral arterial cannula having a balloon to seal said femoral artery;
once in situ in said femoral artery, inflating said femoral arterial cannula balloon to seal said femoral artery to deliver blood flow in a retrograde direction to said limb while simultaneously delivering systemic arterial blood flow;
inserting a femoral venous cannula into a femoral vein of said limb, said femoral venous cannula having a balloon to seal said femoral vein; and
once in situ in said femoral vein, inflating said femoral venous cannula balloon to seal said femoral vein to drain venous blood flow from said limb while simultaneously draining systemic venous blood flow.

2. The method of claim 1 wherein said femoral arterial cannula comprises a low-profile balloon.

3. The method of claim 1 wherein said femoral venous cannula comprises a broad-profile balloon.

4. A femoral arterial cannula comprising:
an elongated hollow cannula body having distal and proximal open ends;
a balloon situated on said body adjacent said distal end;
inflation means connected to said balloon for inflating said balloon;
four blood flow exit ports situated in said cannula body between said balloon and said distal end, two of said distal exit ports in-line with one-another along said cannula body and two other distal exit ports being 180° circumferentially offset from the first two distal exit ports; and
two blood flow exit ports situated in said cannula body between said balloon and said proximal end, said proximal exit ports being 180° circumferentially offset and longitudinally offset from one-another.

5. The femoral arterial cannula of claim 4 wherein said cannula body is an elongated hollow cylindrical body.

6. The femoral arterial cannula of claim 4 wherein said balloon comprises a low-profile balloon.

7. The femoral arterial cannula of claim 4 wherein said cannula body comprises a taper from said distal to said proximal end.

8. The femoral arterial cannula of claim 4 wherein said cannula body comprises a distal single radio-opaque marker.

9. The femoral arterial cannula of claim 4 wherein said cannula body comprises a proximal double radio-opaque marker.

10. The femoral arterial cannula of claim 4 wherein said inflation means comprises:
a small bore tubing connected to said balloon at a first end;
a balloon cuff connected to a second end of said small bore tubing;
a two-way valve connected to said balloon cuff; and
a female luer port connected to said two-way valve.

11. A femoral venous cannula comprising:
an elongated hollow cannula body having distal and proximal open ends;
a balloon situated on said body adjacent said proximal end;
inflation means connected to said balloon for inflating said balloon;
two blood flow entry ports situated in said cannula body between said balloon and said distal end, said distal entry ports being 180° circumferentially offset and longitudinally offset from one-another along said cannula body; and
two blood flow entry ports situated in said cannula body between said balloon and said proximal end, said proximal entry ports being 180° circumferentially offset and longitudinally offset from one-another and 90° offset from said distal entry ports.

12. The femoral venous cannula of claim 11 wherein said elongated hollow cannula body is of various lengths.

13. The femoral venous cannula of claim 11 wherein said cannula body is an elongated hollow cylindrical body.

14. The femoral venous cannula of claim 11 wherein said balloon comprises a broad-profile balloon.

15. The femoral venous cannula of claim 11 wherein said cannula body comprises a taper from said distal to said proximal end.

16. The femoral venous cannula of claim 11 wherein said cannula body comprises a distal single radio-opaque marker.

17. The femoral venous cannula of claim 11 wherein said cannula body comprises a proximal double radio-opaque marker.

18. The femoral venous cannula of claim 11 wherein said inflation means comprises:
a small bore tubing connected to said balloon at a first end;
a balloon cuff connected to a second end of said small bore tubing;
a two-way valve connected to said balloon cuff; and
a female luer port connected to said two-way valve.
